(19) United States
(12) Patent Application Publication
(10) Pub. No.: US 2018/0014732 A1
(43) Pub. Date: Jan. 18, 2018

(54) PHOTOACOUSTIC APPARATUS, IMAGE DISPLAY METHOD, AND PROGRAM

(71) Applicant: CANON KABUSHIKI KAISHA, Tokyo (JP)

(72) Inventors: Takeshi Sekiya, Kawasaki-shi (JP); Kazuhito Oka, Tokyo (JP)

(21) Appl. No.: 15/541,339

(22) PCT Filed: Dec. 25, 2015
(86) PCT No.: PCT/JP2015/006482

§ 371 Date: Jun. 30, 2017

(30) Foreign Application Priority Data
Jan. 7, 2015 (JP) 2015-001683

(51) Int. Cl. A61B 5/00; A61B 5/145

(52) U.S. Cl. CPC A61B 5/0095; A61B 5/72; A61B 5/0082; A61B 5/4848; A61B 5/14542

(57) ABSTRACT

The present invention provides a photoacoustic apparatus by which a redundant time required until presentation of an image to a user can be reduced. A photoacoustic apparatus according to the present invention includes a storage unit configured to hold an electric signal acquired by conversion of a photoacoustic wave occurring from light irradiated to a subject, an input unit configured to designate a region of interest, and an information acquiring unit configured to determine a voxel size based on a size of a region of interest, calculate a subject information distribution in the region of interest by using the voxel size based on the electric signal held in storage unit and cause a display unit to display the subject information distribution.

### TECHNICAL FIELD

The present invention relates to a photoacoustic apparatus which acquires subject information by using a photoacoustic effect.

### BACKGROUND ART

In medical fields, a photoacoustic apparatus has been studied which irradiates pulsed light to a subject, receives photoacoustic waves occurring from the subject due to the light, and analyzes the reception signal of the photoacoustic waves to image subject information.

PTL 1 discloses an apparatus which images subject information regarding a region of interest (ROI) by performing image reconstruction based on a back projection algorithm using a reception signal of photoacoustic waves.

### CITATION LIST

### Patent Literature

PTL 1: Japanese Patent Laid-Open No. 2014-113496

### Non Patent Literature

NPL 1: Nah, Fiona Fui-Hoon. “A study on tolerable waiting time: how long are web users willing to wait?” Behavior & Information Technology 23.3 (2004): 153-163.

### SUMMARY OF INVENTION

### Solution to Problem

In the image reconstruction as disclosed in PTL 1, the time required for the image reconstruction may increase as the number of voxels within an ROI increases. Thus, as the number of voxels within an ROI increases, a longer time may be required until presentation of the corresponding image to a user.

A photoacoustic apparatus according to the present invention includes a storage unit configured to hold an electric signal acquired by conversion of a photoacoustic wave occurring from light irradiated to a subject, an input unit configured to designate a region of interest, and an information acquiring unit configured to determine a voxel size based on a size of a region of interest, calculate a subject information distribution in the region of interest by using the voxel size based on the electric signal held in storage unit and cause a display unit to display the subject information distribution.

Further features of the present invention will become apparent from the following description of exemplary embodiments with reference to the attached drawings.

### BRIEF DESCRIPTION OF DRAWINGS

FIG. 1 is a configuration diagram of a photoacoustic apparatus according to a first exemplary embodiment.

FIG. 2 is a flowchart representing an image display method according to the first exemplary embodiment.

FIG. 3 is a flowchart representing an initial-sound-pressure distribution calculation method according to the first exemplary embodiment.

FIG. 4A illustrates a first user interface example according to the first exemplary embodiment.

FIG. 4B illustrates the first user interface example according to the first exemplary embodiment.

FIG. 4C illustrates the first user interface example according to the first exemplary embodiment.

FIG. 4D illustrates the first user interface example according to the first exemplary embodiment.

FIG. 5A illustrates a second user interface example according to the first exemplary embodiment.

FIG. 5B illustrates the second user interface example according to the first exemplary embodiment.

FIG. 6 illustrates a third user interface example according to the first exemplary embodiment.

FIG. 7 is a configuration diagram of a photoacoustic apparatus according to a second exemplary embodiment.

FIG. 8 is a flowchart representing an optical-absorption coefficient distribution calculation method according to the second exemplary embodiment.

### DESCRIPTION OF EMBODIMENTS

A photoacoustic apparatus may be used to image subject information associated with an optical-absorption coefficient of a subject by analyzing photoacoustic waves. The term “optical-absorption coefficient” here refers to a rate of absorption of optical energy by a biological tissue. For example, subject information originating from photoacoustic waves acquired by a photoacoustic apparatus may be an initial sound pressure, an optical absorption energy density, an optical-absorption coefficient or the like. An optical-absorption coefficient may be acquired for light having a plurality of wavelengths, and the optical-absorption coefficient acquired based on the light having a plurality of wavelengths may be analyzed to acquire the density of a component of the subject. For example, the photoacoustic apparatus may be used to acquire the density ratio between oxyhemoglobin and deoxyhemoglobin so that the oxygen saturation of the biological tissue can be calculated. The density of such components may be subject information which can be acquired by the photoacoustic apparatus.

A photoacoustic apparatus may reconstruct a two- or three-dimensional subject information distribution and generate a slice image or an MIP image from the acquired subject information distribution as required so that the image can be presented to a user (mainly an examiner such as a medical staff). There may be a case where a user needs to observe a specific region of a displayed image in more detail. In this case, the photoacoustic apparatus may reduce the size of each of voxels constructing a subject information distribution to reconstruct the image at finer voxel pitches so that a more detail image may be presented.

However, a voxel size reduced in all of regions of an image displayed to a user may increase the total number of voxels reconstructing the image. Thus, the time required for the reconstruction processing may also increase. Particularly when a three-dimensional subject information distribution is to be reconstructed, a reduced voxel size may significantly increase the number of voxels and thus increase the processing time, compared with the reconstruction of a two-dimension subject information distribution.

Accordingly, a photoacoustic apparatus according to the present invention determines the voxel size for reconstruction based on the size of a designated ROI. Thus, a redundant processing time required for the reconstruction can be reduced.

The term “voxel size” here refers to a length of one side of each of three-dimensional voxels having an equal size and spread all over a space closely. It should be noted that a minimum area at each lattice point of an area to be reconstructed will be called a voxel herein even if the minimum area is two-dimensional. In other words, a two-dimension minimum area which is generally called a pixel will be called a “voxel” in which the concept of a “pixel” is extended three-dimensionally. A method for determining a voxel size will be described in detail in descriptions of exemplary embodiments.

With reference to drawings, exemplary embodiments of the present invention will be described in detail. Like numbers refer to like parts throughout in principle, and repetitive descriptions will be omitted.

### First Exemplary Embodiment

A photoacoustic apparatus according to a first exemplary embodiment is an apparatus which reconstructs an electric signal acquired by conversion of a photoacoustic wave and displays an initial sound pressure distribution.

System Configuration

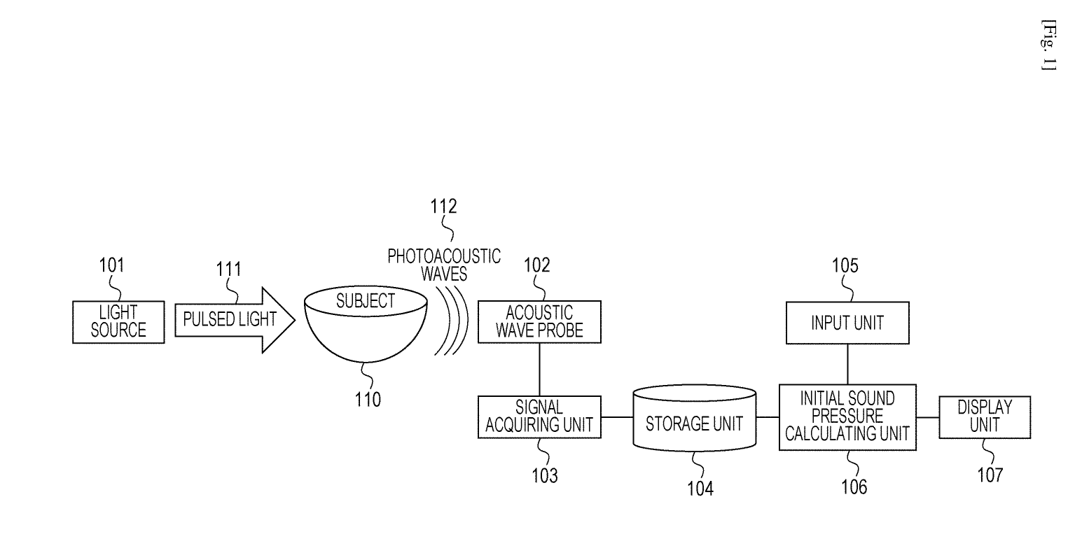

A configuration of the photoacoustic apparatus according to the first exemplary embodiment will be described with reference to FIG. 1.

The photoacoustic apparatus according to the first exemplary embodiment includes a light source 101, an acoustic wave probe 102, a signal acquiring unit 103, a storage unit 104, an input unit 105, an initial sound pressure calculating unit 106, and a display unit 107.

The components will be described below.

Light Source 101

The light source 100 may be a pulsed light source capable of generating pulsed light 111 of nano to micro second-order. The specific pulse width may be in a range of approximately 1 to 100 nanoseconds. The wavelength may be in a range of approximately 400 nm to 1600 nm. Particularly, light having a wavelength in a visible light region (equal to or larger than 400 nm and equal to or smaller than 700 nm) is preferable for imaging a blood vessel near a surface of a living body at a high resolution. On the other hand, light having a wavelength (equal to or higher than 700 nm and equal to or lower than 1100 nm) which is less absorbed by a background tissue of the living body is preferable for imaging a deep part of a living body.

The light source 101 may be a laser. For a measurement using light having a plurality of wavelengths, a laser may be used in which the wavelength of light oscillated by the laser can be converted. In a case where light with a plurality of wavelengths is irradiated to the subject 110, a plurality of lasers which oscillate light with different wavelengths from each other may be used by switching the oscillation wavelength or by irradiating alternately. A plurality of lasers may be handled collectively as a light source.

Various lasers may be used such as a solid laser, a gas laser, a dye laser, a semi-conductor laser. Particularly, a pulsed laser may be used such as an Nd:YAG laser and an alexandrite laser. Alternatively, a Ti:sa laser or OPO (Optical Parametric Oscillators) laser excited by Nd:YAG laser light may be used. A light emitting diode may be used instead of such a laser.

Acoustic Wave Probe 102

The acoustic wave probe 102 converts a photoacoustic wave generated by irradiating pulsed light 111 emitted from the light source 101 to the subject 110 to an electric signal. The acoustic wave probe 102 has one or more acoustic wave transducers and a housing. The acoustic wave transducer may be anything capable of receiving an acoustic wave and converting it to an electric signal, such as a piezoelectric element applying a piezoelectric phenomenon of lead zirconate titanate (PZT), for example, an acoustic wave transducer applying oscillation of light, and a capacitance type acoustic wave transducer such as a CMUT (capacitive micromachined ultrasonic transducer). In a case where a plurality of acoustic wave transducers are provided, they may be arranged within a plane or a curved surface, as in a 1D array, a 1.5D array, a 1.75D array, or a 2D array, for example.

In order to acquire subject information in a wide range, the photoacoustic probe 102 may be configured to mechanically move about the subject 110 by using a scanning mechanism (not illustrated).

In a case where the acoustic wave probe 102 is of a hand-held type, the acoustic wave probe 102 has a grip with which a user can grip the acoustic wave probe 102. An acoustic lens may be provided on the receiving surface of the acoustic wave probe 102. The acoustic wave probe 102 may have a plurality of acoustic wave transducers.

The acoustic wave probe 102 may have an amplifier which amplifies time-series analog signals output from the transducer. In other words, the acoustic wave probe 102 may have a signal acquiring unit 103, which will be described below.

Signal Acquiring Unit 103

The signal acquiring unit 103 is a device which is connected to the acoustic wave probe 102 via a signal line and which performs AD conversion on an electric signal acquired by the acoustic wave probe 102 and writes it in the storage unit 104. The signal acquiring unit 103 may be connected to a light detecting sensor attached to a unit for emitting the pulsed light 111, for example, and emission of the pulsed light 111 may trigger the start of processing of the signal acquiring unit 103 in synchronization.

Storage Unit 104

The storage unit 104 is a medium storing electric signal data having undergone AD conversion. The storage unit 104 may typically be a non-transitory storage medium such as a magnetic disk and a flash memory. The storage unit 104 may be a volatile medium such as a DRAM (dynamic random access memory). It should be noted that a storage medium storing a program is preferably a non-transitory storage medium.

Input Unit 105

The input unit 105 is usable for designating a region of interest by a user. The input unit 105 may include a mouse, a keyboard, and a touch panel. In response to a user's operation thereon, an event is notified to a software program such as an OS running on a CPU (not illustrated) serving as a control unit. For example, a user may operate the input unit 105 so that a GUI (graphical user interface) item displayed on the display unit 107 may be operated for the corresponding input. The input unit 105 may receive an input from a user and transmit the input information to a component such as the initial sound pressure calculating unit 106 in the photoacoustic apparatus. For example, when a user changes an ROI, the input unit 105 notifies the range of the ROI to the initial sound pressure calculating unit.

The input unit 105 in a photoacoustic apparatus of a hand-held type preferably has a function for instructing the driving of the apparatus. The input unit 105 provided in such a hand-held photoacoustic apparatus may be a button-type switch or a foot switch provided on a probe.

It should be noted that a plurality of the input units 105 may be provided as required.

Initial Sound Pressure Calculating Unit 106

The initial sound pressure calculating unit 106 reconstructs an initial sound pressure distribution based on data of an electric signal held in the storage unit 104. The initial sound pressure calculating unit 106 may typically be a processor such as a CPU, GPU (graphics processing unit) or a calculation circuit such as an FPGA (field programmable gate array) chip. It should be noted that the initial sound pressure calculating unit 106 may not only include one processor or calculation circuit but also include a plurality of processors or calculation circuits.

The initial sound pressure calculating unit 106 is capable of calculating an initial sound pressure distribution by using a publicly known reconstruction algorithm such as UBP(universal back-projection), a filtered back projection (FBP), or a model-based method.

It should be noted that processing to be performed by the initial sound pressure calculating unit 106 is stored as a program in the storage unit 104. Then, the CPU serving as a control unit may read out the program stored in the storage unit 104, and the initial sound pressure calculating unit 106 may execute the processing described in the program. According to this exemplary embodiment, the initial sound pressure calculating unit 106 may correspond to an information acquiring unit.

It should be noted that the signal acquiring unit 103 and the initial sound pressure calculating unit 106 may include a common computing element or calculation circuit. In other words, the photoacoustic apparatus according to this exemplary embodiment may have a computing element or calculation circuit functioning as the signal acquiring unit 103 and the initial sound pressure calculating unit 106.

Display Unit 107

The display unit 107 displays an initial sound pressure distribution calculated by the initial sound pressure calculating unit 106. The display unit 107 may be a display such as an LCD (liquid crystal display), a CRT (cathode ray tube), and organic electroluminescence display. The display unit 107 may be provided separately from the photoacoustic apparatus.

Subject 110

Though the subject 110 is not included in the photoacoustic apparatus, it will be described below. A photoacoustic apparatus according to the following embodiment is mainly usable for a diagnosis and a follow-up study of a chemical treatment performed on a blood vessel disease or a malignant tumor of a human or an animal. Therefore, the subject 110 may be may be a living body, and, more specifically, it may be a diagnosis target region such as the breast, the neck, or the abdomen of a human or animal body. For example, in a case where a human body is a measurement target, the target of the light absorber may be oxyhemoglobin or deoxyhemoglobin or a blood vessel containing a large amount of them or a malignant tumor containing many neovessels.

Image Display Method

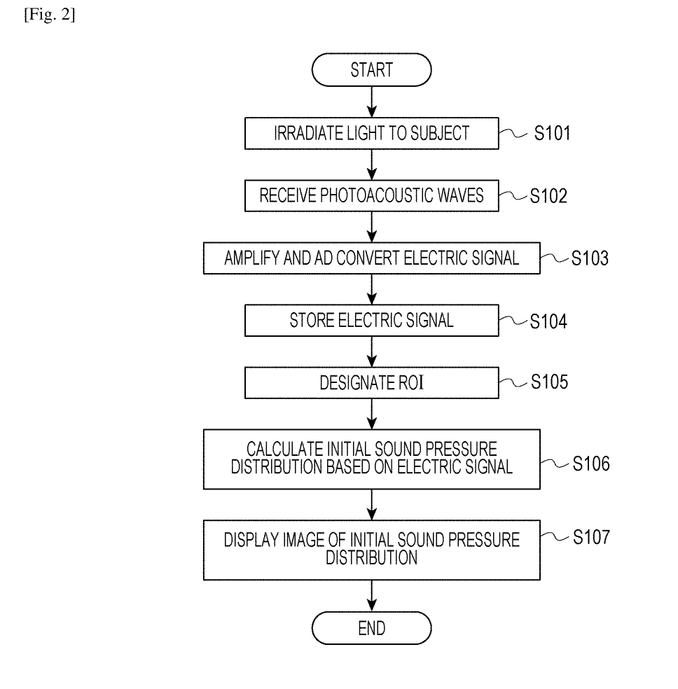

With reference to FIG. 2, a method for displaying an image of an initial sound pressure distribution by using the photoacoustic apparatus according to this exemplary embodiment will be described. It should be noted that a computer including a computing element such as a CPU reads out a program which is stored in the storage unit 104 and in which the image display method is described and causes the photoacoustic apparatus to execute the following image display method.

The light source 101 emits light, and pulsed light 111 is irradiated to the subject 110 (S101). The pulsed light 111 is absorbed by the subject 110, and photoacoustic waves 112 occur due to a photoacoustic effect. The acoustic wave probe 102 receives the photoacoustic waves 112 and outputs time-series analog electric signals (S102). The time-series analog signals output from the signal acquiring unit 103 and acoustic wave probe 102 are collected and undergo amplification processing and AD conversion processing (S103). The signal acquiring unit 103 then stores the digitized time-series electric signals in the storage unit 104 (S104).

Next, the input unit 105 outputs information regarding an ROI designated by a user to the initial sound pressure calculating unit 106 (S105). In other words, a user may use the input unit 105 to designate an ROI. It should be noted that any ROI designation method and any designated region are applicable as long as an ROI can be designated. For example, a user may designate an arbitrary region as an ROI by using the input unit 105 from an optical image of the subject 110 captured by a camera (not illustrated) such as a CCD and displayed by the display unit 107. For example, the initial sound pressure calculating unit 106 may read out a predetermined ROI held in the storage unit 104 and designate a predetermined ROI.

Next, the initial sound pressure calculating unit 106 reads out electric signals from the storage unit 104 and acquires an initial sound pressure distribution of the ROI designated in S105 (S106). The initial sound pressure calculating unit 106 transmits data of the acquired initial sound pressure distribution to the display unit 107 and causes the display unit 107 to display an image of the initial sound pressure distribution (S107).

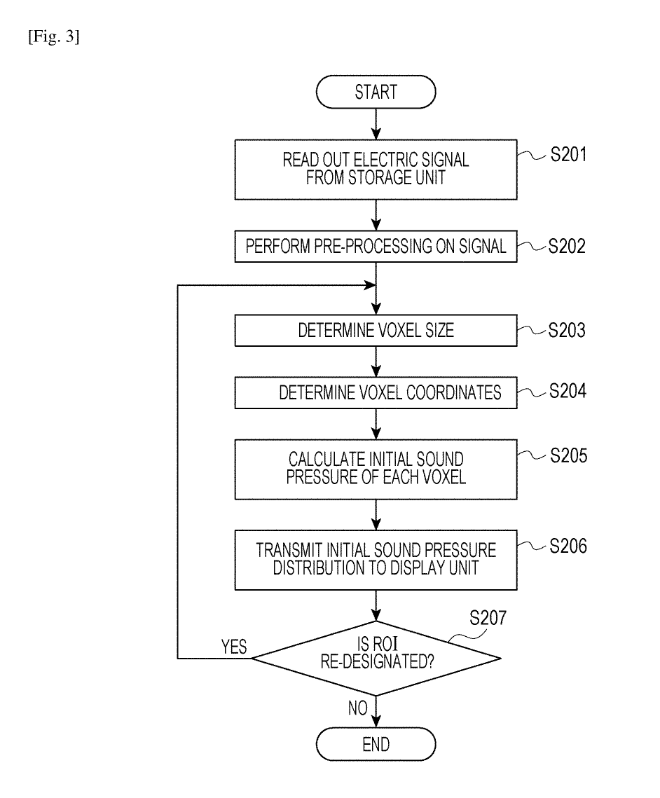

With reference to FIG. 3, step S106 and step S107 to be executed by the initial sound pressure calculating unit 106 will be described in detail. Here, the initial sound pressure calculating unit 106 may apply UBP as a reconstruction scheme, for example.

The initial sound pressure calculating unit 106 starts operating and then reads the data of the electric signals from the storage unit 104 (S201). Next, the initial sound pressure calculating unit 106 performs pre-processing on the electric signals (S202). The pre-processing here refers to processing to be performed on an electric signal independently of the range of the ROI. According to UBP, the initial sound pressure calculating unit 106 performs pre-processing based on Expression (1) below:

$$\begin{matrix}[ Math . 1 ] & \\ b ( {r}_{0} , \overline{t} ) = 2 p ( {r}_{0} , \overline{t} ) - 2 \overline{t} \frac{\partial p ( {r}_{0} , \overline{t} )}{\partial t} & ( 1 )\end{matrix}$$

In this case p(r0,t) is a value in a time t of a signal acquired by an acoustic wave transducer at a position r0, and b(r0,t) is a value acquired by performing pre-processing on p(r0,t).

Next, the initial sound pressure calculating unit 106 determines a voxel size for calculating (reconstructing) an initial sound pressure distribution based on the size of the ROI designated in S105 (S203).

A method for determining the voxel size will be described in detail below.

The initial sound pressure calculating unit 106 may determine the voxel size based on the number of pixels in a display area in which an image of the initial sound pressure distribution is displayed of all display areas of the display unit 107. The display area in which an image of an initial sound pressure distribution is displayed of all display areas will simply be called a “display area”. Having calling a two-dimension and three-dimensional minimum areas to be reconstructed as a “voxel” in unified manner, a minimum area in which information regarding a subject is displayed within a display area will be called a “pixel”.

More specifically, the initial sound pressure calculating unit 106 may determine the voxel size such that the “the number of pixels in the display area×voxel size” can be equal to the size of the ROI designated in S105. For example, a case will be considered in which the size of the ROI designated in S105 is 256 mm×256 mm on an XY plane and the number of pixels corresponding to the XY plane of the display area is 256×256. In this case, the initial sound pressure calculating unit 106 determines the voxel size as 1 mm for satisfying “voxel size=size of ROI (256 mm)÷the number (256) of pixels in display area”. Next, a case will be described in which the pixels having an equal size are spread closely over the display area. Information regarding the number of pixels in the display area may be received by the initial sound pressure calculating unit 106 from the display unit 107 and may be input by a user by using the input unit 105.

A case will be examined in which the aspect ratio of a designate ROI and the aspect ratio of the display area are different. In this case, in order to fit the ROI to the display area, the voxel size is preferably determined with reference to the direction orthogonal to the side of the ROI matching with an outer edge of the display area. In other words, the voxel size is preferably determined such that “the number of pixels in the display area in the orthogonal direction×voxel size” can match with the length of the ROI in the orthogonal direction. Calculating an initial sound pressure distribution at finer voxel pitches than the resolution of the display unit 107 may not easily contribute to an improvement of quality of the image of an initial sound pressure distribution. Therefore, in both of XY directions, determining the voxel size such that number of voxels to be reconstructed can be higher than the number of pixels in the display area increase the amount processing of while not easily increasing the quality of the image.

For example, a case will be examined in which the size of the ROI designated in S105 is 54 mm×128 mm on the XY plane and in which the number of pixels corresponding to the XY plane of the display unit 107 is 256×256. In this case, when the ROI is attempted to be fitted into the display area, the side of the ROI in the X direction is matched with the outer edge of the display area. Accordingly, the initial sound pressure calculating unit 106 determines the voxel size as 0.5 mm to satisfy a condition that “voxel size=the length (128 mm) of the ROI in the Y direction÷the number of pixels (256) in the Y direction”.

Determining the voxel size in accordance with the number of pixels in the display area in this way can reduce the redundant processing time due to reconstruction at voxel pitches each of which is larger than the resolution of the display unit 107 for the designated ROI.

The initial sound pressure calculating unit 106 may determine the voxel size such that the image of the initial sound pressure distribution can be presented within a time period desired by a user. For example, as disclosed in NPL 1, it is known that a user perceives that an apparatus responds instantly as long as the apparatus responds within 0.1 second from an instruction given by the user. Accordingly, the initial sound pressure calculating unit 106 can determine based on information regarding the calculation capability of the photoacoustic apparatus the voxel size with which an initial sound pressure distribution in the ROI designated in S105 can be acquired within 0.1 seconds. This can reduce the stress imposed on a user due to the standby time required from the designation of the range of an ROI to the presentation of the corresponding image. It should be noted that the predetermined time period required for presentation of an image is not limited to 0.1 seconds and may be set by using the input unit 105, for example. The information regarding the calculation capability of the photoacoustic apparatus may be stored in the storage unit 104.

Next, the initial sound pressure calculating unit 106 determines the voxel coordinates based on the range of the ROI designated in step S105 (S204). For example, the range of an ROI may be designated as a square about coordinates (0,0) on the XY plane. When the value of the Z coordinate is equal to 0, the coordinates of the voxel are determined as (−128,−128,0), (−127,−128,0), . . . , (0,−128,0), . . . , (127,−128,0), (−128,−127,0), (−127,−127,0), . . . , (127,127,0).

It should be noted that S203 and S204 may be executed at any time points between S105 and S205.

Next, the initial sound pressure calculating unit 106 calculates the initial sound pressures of the voxels determined in S203 and S204 (S205). According to UBP, the initial sound pressure calculating unit 106 calculates the initial sound pressures by using Expression (2) below.

$$\begin{matrix}[ Math . 2 ] & \\ {p}^{{( b )}_{0}} ( r ) = \frac{{\sum }_{i = 1}^{N} Δ {Ω}_{i} \times b ( {d}_{i} , \overline{t} = \vert {d}_{i} - r \vert )}{{\sum }_{i = 1}^{N} Δ {Ω}_{i}} & ( 2 )\end{matrix}$$

In this case, p(b)0(r) is an initial sound pressure of a voxel to be reconstructed, and N is the number of transducers. ΔΩi is a weight based on a solid angle expecting the ith transducer from the voxel to be reconstructed, which is expressed by using Expression (3).

$$\begin{matrix}[ Math . 3 ] & \\ Δ {Ω}_{i} = \frac{Δ {S}_{i}}{{\vert r - {d}_{i} \vert}^{2}} \cdot [ {n}_{0 i}^{s} \cdot \frac{( r - {d}_{i} )}{\vert r - {d}_{i} \vert} ] & ( 3 )\end{matrix}$$

In this case, di is a position vector of the ith transducer, ΔSi is the area of the ith transducer, and ns0i is a unit normal vector against the plane of the ith transducer for di.

Next, the initial sound pressure calculating unit 106 transmits the initial sound pressures of the calculated voxels to the display unit 107 as an initial sound pressure distribution (S206). In this example, because 256×256×1 voxels are reconstructed, the resulting image can be displayed as 256×256 image as it is. Alternatively, for example, when 256×256×8 voxels are reconstructed, a 256×256 slab MIP (Maximum Intensity Projection) projected in the z direction may be displayed.

Next, the initial sound pressure calculating unit 106 determines whether a notification of re-designation of an ROI has been received from the input unit 105 or not while the image of the initial sound pressure distribution is being displayed on the display unit 107 (S207). If the initial sound pressure calculating unit 106 receives the notification of the re-designation of an ROI from the input unit 105, the processing moves to step S203. The processing in S203 to S206 is executed second time and subsequently based on information regarding the re-designated ROI. It should be noted that the re-designated ROI corresponds to a second region of interest. The voxel size determined in S203 based on the size of the re-designated ROI corresponds to a second voxel size. The initial sound pressure distribution calculated with the second voxel size in S205 corresponds to a second subject information distribution.

As described above, by applying the image display method according to this exemplary embodiment, a redundant processing time for calculating an initial sound pressure distribution can be reduced.

User Interface

Next, an example of a GUI (graphical user interface) according to this exemplary embodiment to be displayed on the display unit 107 will be described.

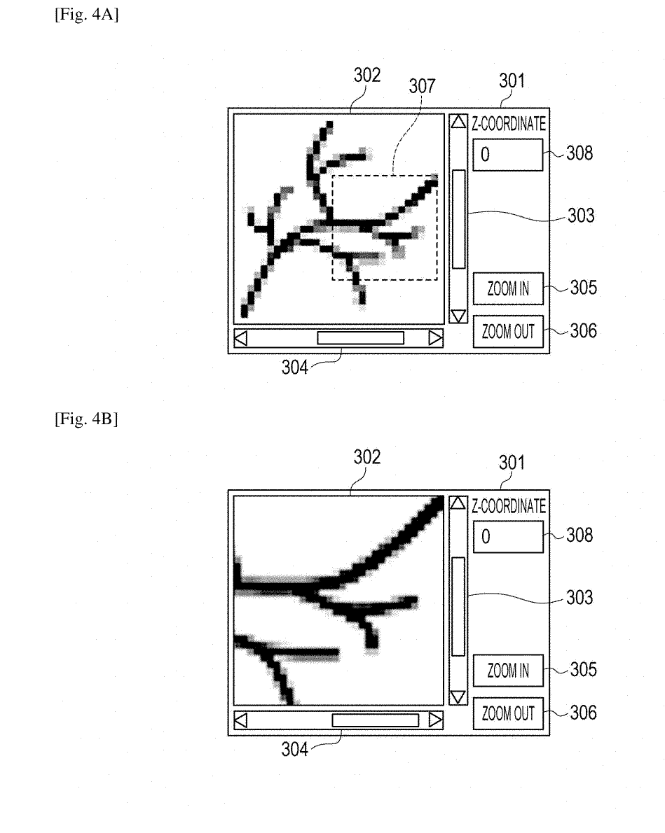

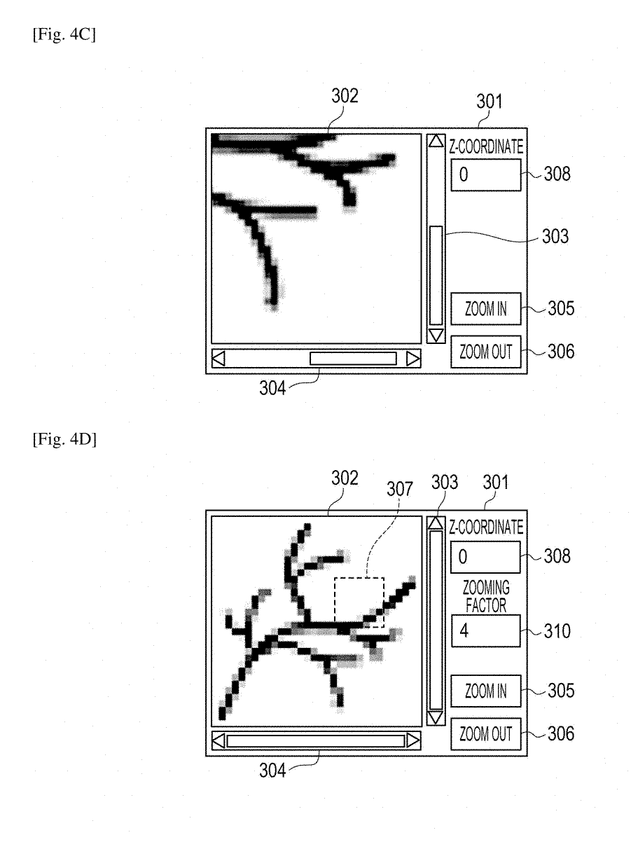

FIG. 4A illustrates an image of an initial sound pressure distribution in the ROI designated in S105. The initial sound pressure distribution illustrated in FIG. 4A is a result acquired by reconstructing with the voxel size determined based on the size of the ROI designated in step S105. This voxel size corresponds to a first voxel size. The initial sound pressure distribution illustrated in FIG. 4A corresponds to a first subject information distribution.

An area 301 is a full display area of the display unit 107. An area 302 is a display area for displaying an initial sound pressure distribution calculated in step S205 among all display areas. A scroll bar 303 and a scroll bar 304 are scroll bars for scrolling an area 307 longitudinally and transversely, respectively. An item 305 and an item 306 are items for displaying the initial sound pressure distribution in an enlarged scale and a reduced scale, respectively. An area 307 is a frame for indicating two-dimensional XY coordinates of an area displayed in an enlarged scale when the item 305 is pressed. An area 308 is a text box area for designating the range of a Z coordinate of an ROI.

A user may use the input unit 105 to operate the scroll bar 303 and scroll bar 304 and moves the area 307 to a desired position. When a user presses the item 305 by using the input unit 105, an area designated in the area 307 and area 308 is displayed in an enlarged scale, and an image of an initial sound pressure distribution illustrated in FIG. 4B is displayed. The initial sound pressure distribution illustrated in FIG. 4B corresponds to a second subject information distribution.

In this case, when the item 305 is pressed, information regarding the ROI based on the area 307 and area 308 is notified from the input unit 105 to the initial sound pressure calculating unit 106. Performing the processing in S203 to S206 based on information regarding the ROI resulting in a change in the image displayed in the area 302. The ROI determined in the area 307 and area 308 corresponds to a second region of interest, and the voxel size determined based on the ROI corresponds to a second voxel size.

According to this exemplary embodiment, the XY plane coordinates designated in the area 307 and the three-dimensional area determined based on the range of the Z coordinate designated in the area 308 corresponds to the second region of interest. Having described that according to this exemplary embodiment, the range of an ROI is designated with the XY coordinates in the area 307 and the range of the Z coordinate separately designated in the area 308, the ROI designation method is not limited thereto. Having described that the ROI range is designated with a rectangular parallelepiped having parallel sides to XYZ axes, the present invention is not limited thereto. An arbitrary area may be defined as an ROI.

When the item 306 is pressed in the state in FIG. 4B, the image displayed in an enlarged scale in FIG. 4B returns to the image in the original scale illustrated in FIG. 4A. In this case, the range of the ROI designated in step S105 is notified to the initial sound pressure calculating unit 106, and an initial sound pressure distribution is calculated again in the same manner. It should be noted that information regarding the previous displayed initial sound pressure distribution may be stored in the storage unit 104, and when an instruction to reduce the scale, information regarding the previously calculated initial sound pressure distribution may be read out from the storage unit 104 and be displayed.

When a user scrolls down the image by using the scroll bar 303 through the input unit 105 in the state in FIG. 4B, the display range may be changed to the one illustrated in FIG. 4C. In this case, the input unit 105 notifies the initial sound pressure calculating unit 106 of that the range of the ROI has been changed. In this case, because the size of the ROI is not changed, the same voxel size as the previous one is determined in S203. However, the voxel coordinates to be reconstructed in S204 are changed. When the range of the ROI is changed, the initial sound pressure calculating unit 106 also recalculates an initial sound pressure distribution. The same is true for a case where the scroll bar 304 is moved. The information regarding the initial sound pressure distribution calculated as in FIG. 4B may be stored in the storage unit 104, and, for the common part of the ROIs in FIG. 4B and FIG. 4C, the previous calculated initial sound pressure distribution may be read out from the storage unit 104, and the values of initial sound pressures may be allocated. Thus, the initial sound pressure of the common part may be acquired without the re-calculation.

For instruction of the enlargement and reduction, the present invention is not limited to the pressing of the item 305 and item 306, but any method may be applied. For example, an enlargement and a reduction may be instructed in accordance with the amount of rotation of a wheel of a mouse serving as the input unit 105. Also for designation of the display range of the area 302 or instruction of a movement of the area 307, the present invention is not limited to the method using the scroll bar 303 and the scroll bar 304, but any method may be applied therefor. For example, the area 302 or the area 307 may be moved by a moving amount from a starting point at a position where a drag is started by the input unit 105 being a mouse and to an end point at a position where the drag ends.

In the examples illustrated in FIGS. 4A to 4C, an image to be displayed in the area 302 is changed by enlarging or reducing by a predetermined scale of enlargement. For that, information regarding voxel sizes corresponding to the predetermined scale of enlargement may be prestored in the storage unit 104, and the initial sound pressure calculating unit 106 may read out the predetermined voxel size corresponding to the predetermined scale of enlargement in step S203. In the examples illustrated in FIGS. 4A to 4C, the predetermined scale of enlargement is set to 2×. The read predetermined voxel size corresponds to the second voxel size.

As illustrated in FIG. 4D, the GUI may have an area 310 which is a text box area in which the scale of enlargement is designated. In other words, the GUI may be configured such that the number to be entered to the area 310 may be changed to change the scale of enlargement in response to the pressing on the item 305. The size of the area 307 may be automatically changed in accordance with the scale of enlargement input to the area 310. The initial sound pressure calculating unit 106 determines the voxel size based on the number input to the area 310 through the input unit 105. In this case, the initial sound pressure calculating unit 106 may divide the previously calculated voxel size by the designated scale of enlargement to determine a new voxel size. Here, the new voxel size corresponds to the second voxel size.

In the description of this exemplary embodiment, the term “enlargement and reduction” is used for convenience. However, the term “enlargement and reduction” does not simply refer to processing of stretching an image but actually refers to re-calculation of the initial sound pressure distribution by changing the voxel size.

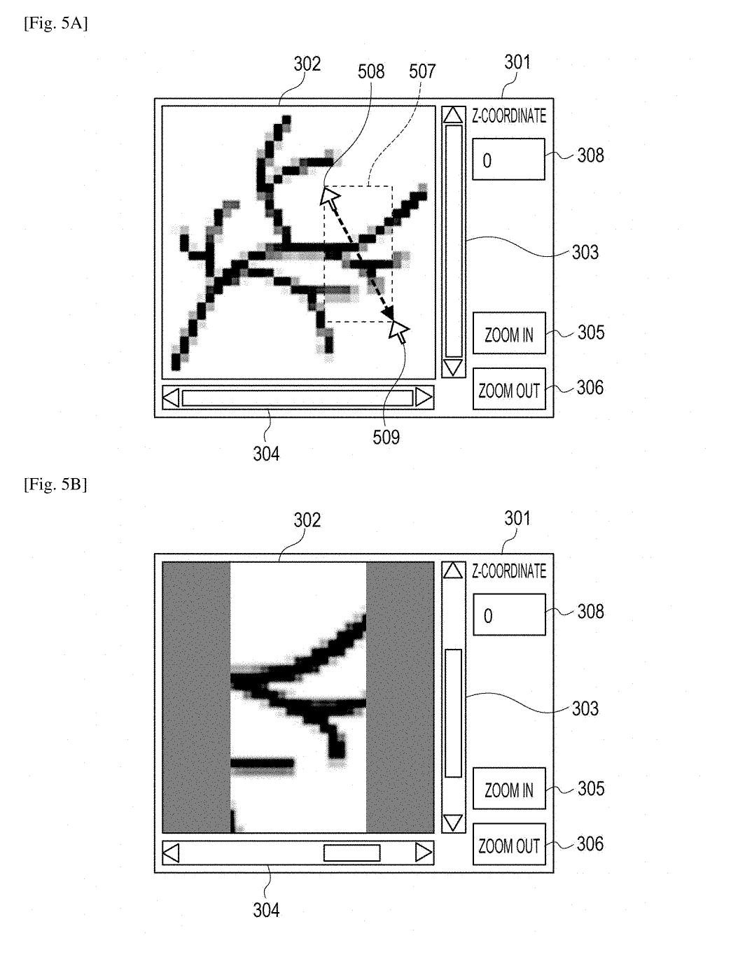

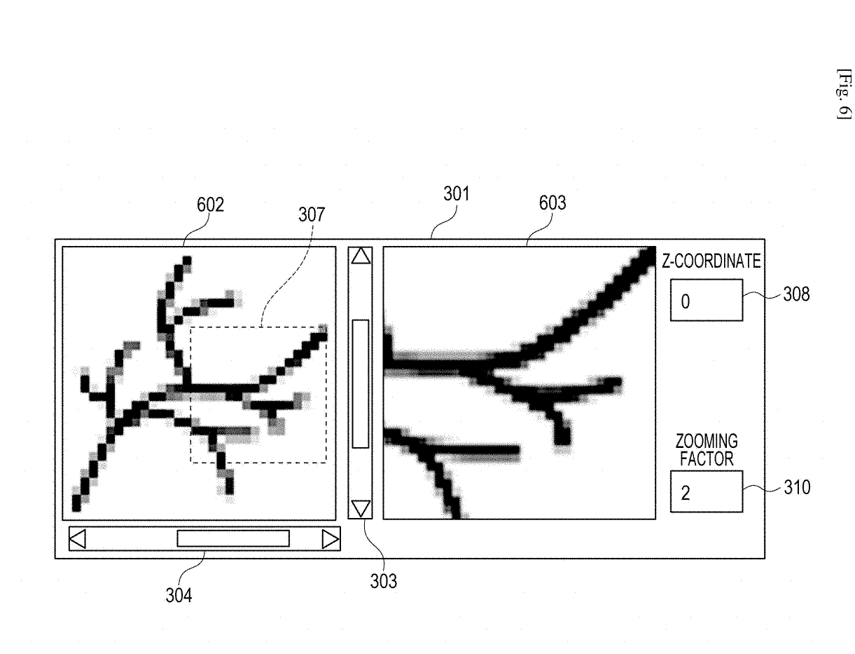

A user may designate an arbitrary area from an image of the initial sound pressure distribution displayed in the display area to set the area as the second region of interest to be re-designated. This example will be described with reference to FIGS. 5A and 5B. A cursor 508 corresponds to a point where a user starts a mouse drag, a cursor 509 corresponds to a point where a user ends the mouse drag, and an area 507 corresponds to an area selected by the mouse drag. As illustrated in FIG. 5A, after the area 507 is designated and when the item 305 is pressed, the image is enlarged as illustrated in FIG. 5B.

In this case, it is assumed, for example, that the ROI size for display in the area 302 in FIG. 5A is 256 mm×256 mm and that the coordinates with the cursor 508 and the cursor 509 are (0,0), and (64,128), respectively, and the size of the area 507 is 64 mm×128 mm. In this case, the initial sound pressure calculating unit 106 determines 0.5 mm as the voxel size so as to satisfy a condition “voxel size=length (128 mm) of the ROI in the vertical direction of the paper surface÷the number of pixels (256) of the display area”.

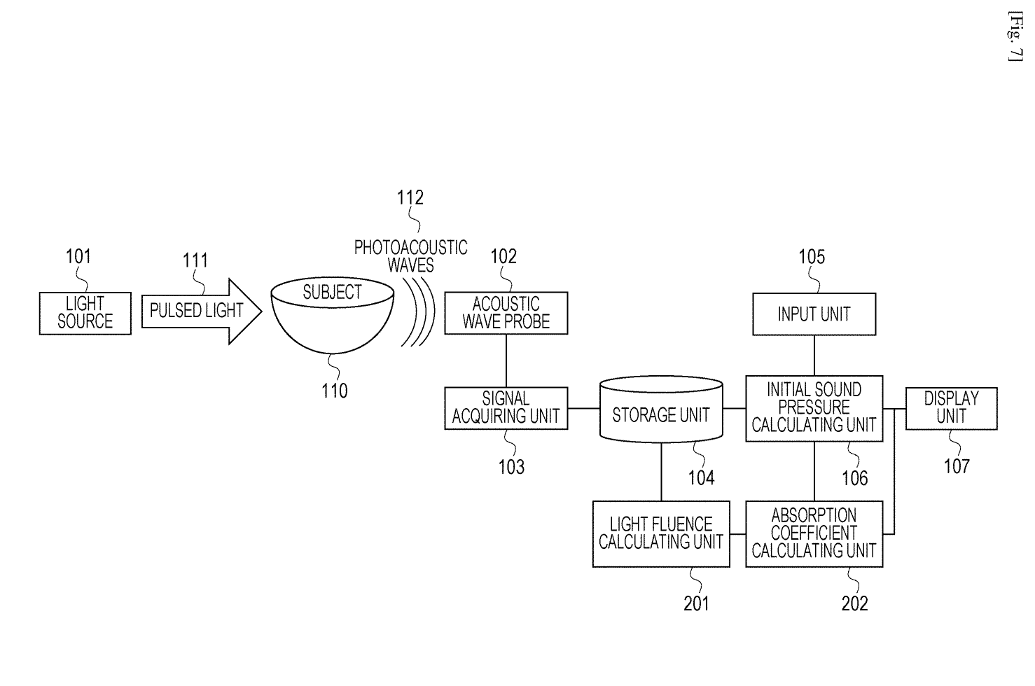

Though FIGS. 4A to 4C and FIGS. 5A and 5B illustrate one area for displaying the initial sound pressure distribution in the display unit 107, images of initial sound pressure distributions before and after an enlargement may be displayed as illustrated in FIG. 6. An area 602 corresponds to a display area for displaying an initial sound pressure distribution before an enlargement. An area 603 corresponds to a display area for displaying an enlarged image of the image in the area 307. According to the method described with reference to FIG. 6, a user can check an enlarged image in the area 603 while checking the area 307 subject to the enlargement in the whole image in the area 602. A user can operate the scroll bar 303 and 304 through the input unit 105 to move the area 307 and in real time check in the area 603 the enlarged image in the area 307. It should be noted that an enlarged image may be generated without an enlargement instruction when the size or position of the area 307 is changed in order to present the images before and after an enlargements side by side as illustrated in FIGS. 5A and 5B. The images before and after an enlargement may be displayed side by side on different displays. In this case, the plurality of displays may be handled as the display unit 107. Though initial sound pressure distributions before and after an enlargement are displayed side by side in FIG. 6, they may be displayed by placing one over the other.

Having described the case a voxel size is always changed when an ROI is designated according to this exemplary embodiment, the present invention is not limited to this aspect. The photoacoustic apparatus according to this exemplary embodiment may be configured in which one of a variable mode which allows change of a voxel size and an invariable mode which does not allow change of a voxel size may be selected through the input unit 105, for example. When a user selects the variable mode, the aforementioned image display method according to this exemplary embodiment may be executed. On the other hand, when the invariable mode is selected, because the voxel size is not changed, the resolution of the resulting enlarged image may decrease. However, because the time required for the processing decreases, the image may be displayed more quickly. Thus, because one of the variable mode which allows display of an image within an ROI with a high resolution (high quality) and the invariable mode which allows display of the image within the ROI in real time can be selected, various user's needs may be satisfied.

The example has been described in which the voxel size is set in accordance with the size of a designated ROI according to this exemplary embodiment to reduce a redundant processing time. However, other processing methods than the voxel size setting may be changed in accordance with the size of a designated ROI. For example, the reconstruction algorithm may be changed in accordance with the size of a designated ROI. When an ROI having a predetermined size or larger is set, a time domain reconstruction or a Fourier domain reconstruction requiring a lower amount of processing may be executed. When an ROI having a predetermined size or smaller is set, a model-based reconstruction may be executed by which a high quality image can be acquired though it requires a higher amount of processing.

### Second Exemplary Embodiment

System Configuration

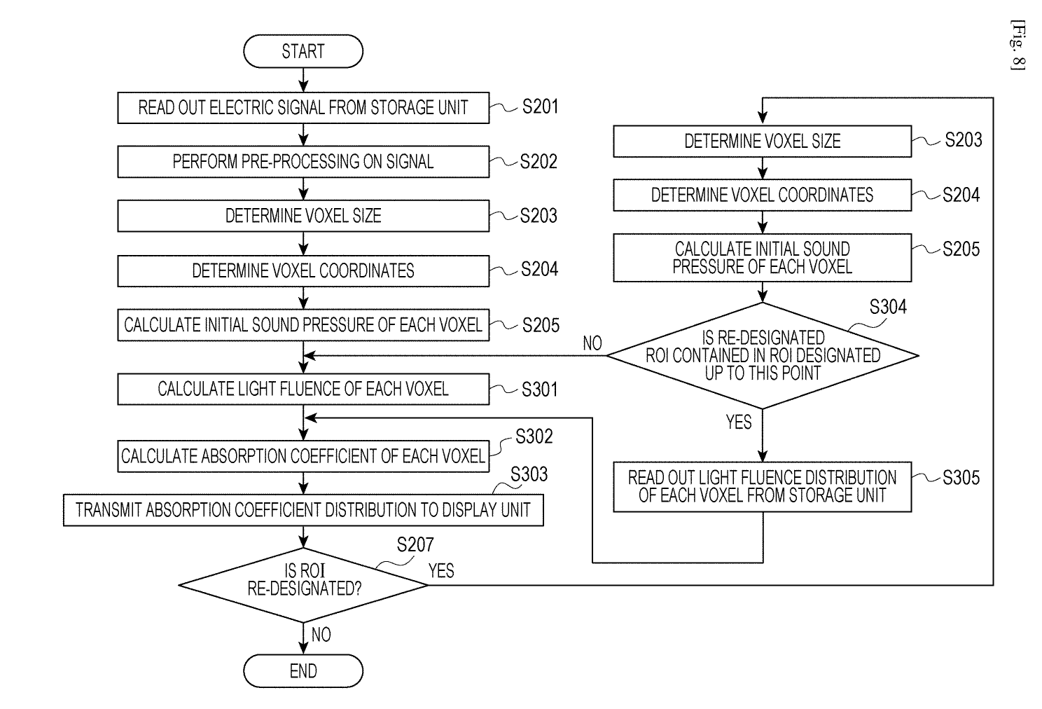

With reference to FIG. 7, a configuration of a photoacoustic apparatus according to a second exemplary embodiment will be described. Like numbers refer to like parts in FIG. 1 and FIG. 7, and detail repetitive descriptions will be omitted.

The photoacoustic apparatus according to the second exemplary embodiment includes a light source 101, an acoustic wave probe 102, a signal acquiring unit 103, a storage unit 104, an input unit 105, an initial sound pressure calculating unit 106, a display unit 107, a light fluence calculating unit 201, and an absorption coefficient calculating unit 202. The photoacoustic apparatus according to the second exemplary embodiment is different from that of the first exemplary embodiment in that the light fluence calculating unit 201 and the absorption coefficient calculating unit 202 are further provided.

The light fluence calculating unit 201 acquires how the intensity of pulsed light 111 irradiated to a subject 110 distributes within the subject based on the intensity of the pulsed light 111 read out from the storage unit 104. Details of processing to be performed by the light fluence calculating unit 201 will be described below.

It has been known that a sound pressure p₀ of acoustic waves caused by a photoacoustic effect can be expressed by using the following Expression (4):

p₀=μₐ*Γ*Φ (4)

where μₐ is an optical-absorption coefficient, Γ is a constant depending on the type of a subject and is called a Gruneisen coefficient, and Φ represents a light fluence.

The absorption coefficient calculating unit 202 calculates an absorption coefficient distribution based on an initial sound pressure distribution acquired by the initial sound pressure calculating unit 106 and a light fluence distribution acquired by the light fluence calculating unit 201 in accordance with Expression (4).

The light fluence calculating unit 201 or the absorption coefficient calculating unit 202 may typically use a processor such as a CPU and a GPU (Graphics Processing Unit), and a calculation circuit such as an FPGA (Field Programmable Gate Array) chip. The light fluence calculating unit 201 or the absorption coefficient calculating unit 202 may include a plurality of processors and calculation circuits instead of one processor and one calculation circuit.

The processing to be performed by the light fluence calculating unit 201 or the absorption coefficient calculating unit 202 is stored as a program in the storage unit 104. A CPU serving as a control unit may read a program stored in the out storage unit 104, and the light fluence calculating unit 201 or the absorption coefficient calculating unit 202 may execute the processing described in the program. According to this exemplary embodiment, the light fluence calculating unit 201 or the absorption coefficient calculating unit 202 corresponds to an information acquiring unit. The initial sound pressure calculating unit 106, the light fluence calculating unit 201, and the absorption coefficient calculating unit 202 may include a common computing element or calculation circuit. In other words, the photoacoustic apparatus according to this exemplary embodiment may include a computing element or calculation circuit which may implement functions of the initial sound pressure calculating unit 106, the light fluence calculating unit 201, and the absorption coefficient calculating unit 202.

Image Display Method

Next, an image display method according to this exemplary embodiment will be described. Particularly, processing to be performed by the initial sound pressure calculating unit 106, the light fluence calculating unit 201, and the absorption coefficient calculating unit 202 will be described in detail. FIG. 8 illustrates a flow of the image display method according to this exemplary embodiment. Like numbers refer to like steps in FIG. 3 and FIG. 8, and the detail repetitive description will be omitted.

The light fluence calculating unit 201 calculates the light fluence of each voxel by using the size and coordinates of the voxel determined in S203 and S204 and acquires a light fluence distribution in the ROI designated in S105 (S301). The light fluence calculating unit 201 further stores the data regarding the light fluence distributions calculated in S301 in the storage unit 104 for use in S304, which will be described below.

For example, the light fluence calculating unit 201 can calculate a light fluence distribution by numerically solving a transport equation or a diffusion equation expressing a behavior of optical energy in a medium in which light is absorbed and is scattered. This numerically solving method may be a finite element analysis, a difference method, a Monte Carlo method or the like. The light fluence distribution acquired based on the calculation method corresponds to a first light fluence distribution.

Next, the absorption coefficient calculating unit 202 calculates an optical-absorption coefficient distribution in the ROI based on the initial sound pressure distributions and light fluence distributions in the ROI designated in S105 by using Expression (4) (S302). The optical-absorption coefficient distribution calculated here corresponds to a first subject information distribution. The absorption coefficient calculating unit 202 transmits data regarding the optical-absorption coefficient distribution calculated in S302 to the display unit 107 (S303). The display unit 107 receives the data regarding the optical-absorption coefficient distribution and displays an image representing the optical-absorption coefficient distribution.

Next, as described above, when an ROI is re-designated, the processing in S203 to S205 is executed based on information regarding the re-designated ROI. The information regarding an ROI input through the input unit 105 is stored in the storage unit 104 for use in S304, which will be described below.

Next, the light fluence calculating unit 201 determines whether the re-designated ROI is contained in the previously designated ROI or not (S304). More specifically, according to this exemplary embodiment, the light fluence calculating unit 201 determines whether the ROI re-designated in S207 is contained in the ROI designated in S105 or not.

If the re-designated ROI is contained in the ROI designated in S105, the processing moves to S305. The inventors have found that a reduced voxel size has a small influence on the prevision of calculation of a light fluence distribution, compared with a reduced voxel size in calculation of an initial sound pressure distribution. Accordingly, if the re-designated ROI is contained in the previously designated ROI, the light fluence calculating unit 201 may acquire a light fluence distribution in the re-designated ROI based on the light fluence distribution in the previously designated ROI, independently from the size of the re-designated ROI. The light fluence calculating unit 201 may allocate a previously acquired light fluence distribution as the light fluence distribution in the re-designated ROI. Alternatively, the light fluence calculating unit 201 may interpolate a previously acquired light fluence distribution to the voxel size corresponding to the re-designated ROI to acquire the light fluence distribution in the re-designated ROI. This can significantly reduce the time required for acquiring a light fluence distribution without significantly reducing the accuracy of the light fluence distribution. In other words, this method can eliminate the necessity for acquiring a light fluence distribution in at least a partial area of a re-designated ROI if any by calculating a light propagation.

The light fluence calculating unit 201 acquires a light fluence distribution in the re-designated ROI based on the light fluence distribution in the ROI designated in S105 held in the storage unit 104 (S304). Thus, the processing for calculating the light fluence distribution in the re-designated ROI in S207 can be omitted. The light fluence distribution acquired in this step corresponds to a second light fluence distribution. A light fluence distribution is calculated for a part not contained in the previously designated ROI of the re-designated ROI. In this case, the light fluence calculating unit 201 may calculate a light fluence distribution in the re-designated ROI by using the light fluence distribution in the previously designated ROI as an initial condition.

On the other hand, if the re-designated ROI is not contained in the ROI designated in S105, the processing moves to S301 where the light fluence calculating unit 201 calculates a light fluence distribution in the re-designated ROI by using the voxel size determined in S203 based on the re-designated ROI (S301).

The absorption coefficient calculating unit 202 then calculates an optical-absorption coefficient distribution in the re-designated ROI based on the initial sound pressure distribution and light fluence distribution in the re-designated ROI by using Expression (4) (S302). The optical-absorption coefficient distribution calculated here corresponds to a second subject information distribution. There may be a case where the voxel size applied to the calculation of the initial sound pressure distribution is different from the voxel size applied to the calculation of the light fluence distribution. In this case, the absorption coefficient calculating unit 202 may be required to sample the value of each voxels for the light fluence distribution to the voxel size applied to the calculation of the initial sound pressure distribution. The absorption coefficient calculating unit 202 therefore may project the value of each of the voxels of the light fluence distribution to the voxels applied to the calculation of the initial sound pressure distribution without changing the values. The absorption coefficient calculating unit 202 may perform a complementary calculation on the values of the voxels of the light fluence distribution and project the results to the voxels applied to the calculation of the initial sound pressure distribution.

The absorption coefficient calculating unit 202 transmits the data regarding the optical-absorption coefficient distribution in the re-designated ROI to the display unit 107, and the display unit 107 is caused to display it (S303).

As described above, the image display method of this exemplary embodiment can reduce a redundant time required for calculating a light fluence distribution in a re-designated ROI and at the same time calculate an optical-absorption coefficient distribution highly accurately.

Having described that according to this exemplary embodiment the voxel size for acquiring a light fluence distribution by performing a calculation is determined based on the size of an ROI, like the calculation of an initial sound pressure distribution, the method for calculating the voxel size applied for calculating a light fluence distribution is not limited thereto. As described above, because the accuracy of calculation of a light fluence distribution is not easily influenced by a voxel size, a predetermined voxel size may be used for calculating a light fluence distribution.

The light fluence calculating unit may acquire a light fluence distribution by using a voxel size larger than a voxel size used for acquiring an initial sound pressure distribution. In the calculation of light propagation of a light fluence distribution, when a given voxel size is smaller than an average free path, the accuracy is less influenced by a reduced voxel size. Therefore, the light fluence calculating unit may calculate a light fluence distribution with a voxel size equal to or larger than an average free path. For example, when a subject is a living body, the light fluence calculating unit may calculate a light fluence distribution with a voxel size equal to or larger than 0.1 mm independently from the size of the ROI. The light fluence calculating unit may calculate a light fluence distribution with a voxel size equal to or larger than 0.5 mm independently from the size of the ROI.

Even when a re-designated ROI is contained in the previously designated ROI, a light fluence distribution may be acquire by performing a light propagation calculation.

The absorption coefficient calculating unit 202 may use light having a plurality of wavelengths according to the aforementioned method to calculate a plurality of optical-absorption coefficient distributions corresponding to the wavelengths. The absorption coefficient calculating unit 202 may calculate a component density distribution of a subject from a plurality of optical-absorption coefficient distributions corresponding to the wavelengths as the first and second subject information distributions instead of the optical-absorption coefficient distribution. Particularly, when main light absorbents in a subject are oxyhemoglobin and deoxyhemoglobin oxidation, the oxygen saturation SO2 can be expressed as a component density by using the following Expression (5).

$$\begin{matrix}[ Math . 4 ] & \\ {SO}_{2} = \frac{- \frac{{μ}_{a} ( {λ}_{1} )}{{μ}_{a} ( {λ}_{2} )} {ɛ}_{de} ( {λ}_{2} ) + {ɛ}_{de} ( {λ}_{1} )}{- \frac{{μ}_{a} ( {λ}_{1} )}{{μ}_{a} ( {λ}_{2} )} \{ {ɛ}_{de} ( {λ}_{2} ) + {ɛ}_{ox} ( {λ}_{2} ) \} + \{ {ɛ}_{de} ( {λ}_{1} ) + {ɛ}_{ox} ( {λ}_{1} ) \}} & ( 5 )\end{matrix}$$

In this case, μₐ(λ) is an optical-absorption coefficient with a wavelength λ, εₒₓ(λ)*ε_{de} (λ) is an optical-absorption coefficient of oxyhemoglobin and deoxyhemoglobin with the wavelength λ. Because εₒₓ(λ)*ε_{de}(λ) is known, the absorption coefficient calculating unit 202 may calculate an oxygen saturation distribution from a plurality of optical-absorption coefficient distributions corresponding to the wavelengths by using Expression (5).

As described above, by applying the method of this exemplary embodiment for acquiring optical-absorption coefficient distributions corresponding to a plurality of wavelengths, a plurality of light propagation calculations for light fluence distributions can be omitted. Thus, a redundant processing time can be reduced.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-001683, filed Jan. 7, 2015, which is hereby incorporated by reference herein in its entirety.

1. A photoacoustic apparatus comprising:
a storage unit configured to store an electric signal acquired by conversion of a photoacoustic wave occurring from light irradiated to a subject;
an information acquiring unit configured to:
set a first region of interest on the basis of information generated based on a designation of a user;
set a first voxel size on the basis of information on the first region of interest;
calculate, in the first voxel size, a first subject information distribution in the first region of interest on the basis of the electric signal stored in the storage unit; and
cause a display unit to display a first image of the first subject information distribution.

2. The photoacoustic apparatus according to claim 1, wherein the information acquiring unit is configured to set the first voxel size on the basis of the information on the first region of interest and information on a number of pixels in a display area of the display unit for the first subject information distribution.

3. The photoacoustic apparatus according to claim 1, wherein the information acquiring unit is configured to set the first voxel size such that the first subject information distribution can be calculated within 0.1 seconds.

4. The photoacoustic apparatus according to claim 1, wherein the information acquiring unit is configured to calculate at least one of an initial sound pressure distribution, an optical absorption energy density distribution, an optical-absorption coefficient distribution, and a distribution of a density of a component of the subject, as the first subject information distribution.

5. The photoacoustic apparatus according to claim 1, wherein the information acquiring unit is configured to:
calculate, in the first voxel size, a first initial sound pressure distribution in the first region of interest based on the electric signal;
calculate, in a voxel size smaller than the first voxel size, a first light fluence distribution of the light in the first region of interest; and
calculate an optical-absorption coefficient distribution in the first region of interest on the basis of information on the first initial sound pressure distribution and information on the first light fluence distribution, as the first subject information distribution.

6. The photoacoustic apparatus according to claim 1, wherein the information acquiring unit is configured to:
set a second region of interest on the basis of information generated based on a designation of the user when the first image is displayed;
set a second voxel size on the basis of information on the second region of interest;
calculate, in the second voxel size, a second subject information distribution in the second region of interest on the basis of the electric signal stored in the storage unit; and
cause the display unit to display a second image of the second subject information distribution.

7. The photoacoustic apparatus according to claim 6, wherein if the second region of interest is contained in the first region of interest, the information acquiring unit is configured to:
calculate, in the first voxel size, a first initial sound pressure distribution in the first region of interest based on the electric signal;
calculate a first light fluence distribution of the light in the first region of interest;
calculate a first optical-absorption coefficient distribution in the first region of interest as the first subject information distribution on the basis of information on the first initial sound pressure distribution and information on the first light fluence distribution;
wherein the storage unit is configured to store the information on the first light fluence distribution,
wherein the information acquiring unit is configured to:
calculate, in the second voxel size, a second initial sound pressure distribution in the second region of interest on the basis of the electric signal;
calculate a second light fluence distribution of the light in the second region of interest on the basis of the information on the first light fluence distribution stored in the storage unit; and
calculate a second optical-absorption coefficient distribution in the second region of interest as the second subject information distribution on the basis of information on the second initial sound pressure distribution and information on the second light fluence distribution.

8. The photoacoustic apparatus according to claim 7, wherein the information acquiring unit is configured to acquire information on the first light fluence in the second region of interest stored the storage unit as the information on the second light fluence distribution in the second region of interest.

9. The photoacoustic apparatus according to claim 7, wherein the information acquiring unit is configured to acquire the information on the second light fluence distribution by interpolating the first light fluence distribution.

10. The photoacoustic apparatus according to claim 1, wherein the information acquiring unit is configured to:
set the second voxel size smaller than the first voxel size in a case where the size of the second region of interest is smaller than the first region of interest;
set the second voxel size larger than the first voxel size in a case where the size of the second region of interest is larger than the first region of interest.

11. The photoacoustic apparatus according to claim 1, further comprising:
a light source configured to radiate the light; and
an acoustic wave probe configured to convert a photoacoustic wave occurring from the light irradiated to the subject to the electric signal.

12. A photoacoustic apparatus comprising:
a storage unit configured to store an electric signal acquired by conversion of a photoacoustic wave occurring from light irradiated to a subject,
an information acquiring unit configured to:
set a first scale of a first image on the basis of information generated based on a designation of a user,
set a first voxel size on the basis of information on the first scale of the first image, and
calculate, in the first voxel size, a first subject information distribution on the basis of the electric signal stored in the storage unit,
a display controlling unit configured to cause a display unit to display, on the first scale, the first image of the first subject information distribution.

13. The photoacoustic apparatus according to claim 12, wherein
the information acquiring unit is configured to:
set a second scale on the basis of information generated based on a designation of the user when the first image is displayed;
set a second voxel size on the basis of information on the second scale;
calculate, in the second voxel size, a second subject information distribution on the basis of the electric signal stored in the storage unit; and
cause the display unit to display, on the second scale, a second image of the second subject information distribution.

14. An image display method comprising:
setting a region of interest on the basis of information generated based on a designation of a user;
setting a voxel size on the basis of information on the region of interest;
calculating, in the voxel size, a subject information distribution in the region of interest on the basis of an electric signal acquired by conversion of a photoacoustic wave occurring from light irradiated to a subject; and
displaying an image of the subject information distribution.

15. An image display method comprising:
setting a scale of an image on the basis of information generated based on a designation of a user;
setting a voxel size on the basis of information on the scale of the image;
calculating, in the voxel size, a subject information distribution on the basis of an electric signal acquired by conversion of a photoacoustic wave occurring from light irradiated to a subject; and
displaying, on the scale, an image of the subject information distribution.

16. An image display method comprising:
displaying a first image originating from a photoacoustic wave occurring from light irradiated to a subject;
setting a region of interest within the first image on the basis of information generated based on a designation of a user; and
displaying a higher quality second image in the region of interest than the first image, the second image originating from the photoacoustic waves.

17. A non-transitory computer-readable storage medium storing a program causing a computer to execute the image display method according to claim 14.

18. A non-transitory computer-readable storage medium storing a program causing a computer to execute the image display method according to claim 15.

19. A non-transitory computer-readable storage medium storing a program causing a computer to execute the image display method according to claim 16.
